(19) United States
(12) Patent Application Publication
(10) Pub. No.: US 2013/0115630 A1
(43) Pub. Date: May 9, 2013

(54) AUTOMATED MULTIPLEX IMMUNOASSAY

(71) Applicants: Paul Shore, Sunniside (GB); Louisa Poole, Newcastle Upon Tyne (GB); Justine Reed, Bedlington (GB)

(72) Inventors: Paul Shore, Sunniside (GB); Louisa Poole, Newcastle Upon Tyne (GB); Justine Reed, Bedlington (GB)

(21) Appl. No.: 13/292,808

(22) Filed: Nov. 9, 2011

(51) Int. Cl. G01N 33/566; G01N 33/53

(52) U.S. Cl. 435/7.24; 435/7.9

(57) ABSTRACT

Embodiments relate generally to an automated multiplex immunoassay for qualitatively and/or quantitatively detecting a plurality of targets on a tissue sample. Kits and apparatuses for practicing such assays are also provided.

### TECHNICAL FIELD

The present invention relates generally to an automated multiplex immunoassay for qualitatively and/or quantitatively detecting a plurality of targets on a tissue sample. The invention also relates to kits and apparatuses for practicing such assays.

### BACKGROUND

Immunohistochemistry (IHC) employs specific binding agents, such as antibodies, to detect an antigen of interest (a biomarker) that may be present in a tissue sample. IHC has become an important diagnostic tool to diagnose a particular disease state or condition. It is also widely used as a basic research tool for investigating the presence and distribution of a biomarker in a tissue sample. IHC has many advantages over traditional methods of measuring protein extracted from whole tissue, including the ability to localize a biomarker of interest to a particular cell type or tissue region.

From a clinical perspective, IHC is routinely performed on preserved (e.g., formalin-fixed), paraffin-embedded tissue samples, which can be archived and re-analyzed up to decades later.

Traditional IHC methods have evolved over the years to allow for the detection of two or more biomarkers in a tissue sample. Such methods utilize multiple primary antibodies that bind to the biomarkers of interest and detection in a sequence of steps to achieve multiple labeling on the same tissue sample. However, detecting multiple biomarkers by IHC is time consuming and is therefore seldom used for clinical diagnoses. For instance, a double IHC stain could require 30 to 50 steps and a triple IHC stain could take more than 50 steps, depending on the complexity of the immunoassay. Nevertheless, the ability to detect multiple biomarkers in a tissue sample would provide a more practical approach for pathologists and clinicians, particularly where there is limit tissue available, as is often the case when using biopsy material.

US patent publication 2010/0047825A1 provides immunoassay “reagents and methods of using the reagents, for example, on automated staining devices, that facilitate detection of two or more antigens in a sample simply and efficiently.” [See Abstract] This publication fails to disclose a fully automated method that performs both antigen retrieval and immunostaining on a single instrument. Further, the described method involves the application of a single cocktail of labeled secondary antibodies to a tissue sample labeled with primary antibodies. This method therefore requires that at least two secondary antibodies have similar buffering requirements, limiting the application, overall sensitivity, and/or specificity of the detection method.

Embodiments described herein overcome or alleviate these and other deficiencies in the art.

### SUMMARY OF THE INVENTION

In a first aspect, there is provided a method of detecting a plurality of epitopes in a biological tissue sample, the method comprising, in order, the following steps:
(a) providing a cross-section of a tissue sample attached to a solid substrate; wherein the tissue sample is embedded in an embedding medium;
(b) removing the embedding medium from the tissue cross-section of (a);
(c) incubating the tissue sample with two or more primary binding agents for a period of time sufficient to allow the two or more primary binding agents to bind to the plurality of epitopes, wherein two or more of the primary binding agents bind to different epitopes;
(d) incubating the tissue sample with two or more secondary binding agents for a period of time sufficient to allow the two or more secondary binding agents to bind to the primary binding agents, wherein each of the two or more of the secondary binding agents binds to a different primary binding agent and wherein two or more of the secondary binding agents comprise different detectable labels; and
(e) detecting the presence of the different detectable labels on the tissue sample, thereby distinguishing between the plurality epitopes;
   wherein progress from step (b) to (e) is automated and wherein the two or more primary binding agents and/or the two or more secondary binding agents are a cocktail of the binding agents.

In some embodiments, methods are implemented under conditions to allow or promote a certain goal. Such conditions are known to those of skill in the art, including, but not limited to, temperature, time, pH, density or viscosity, and/or reaction volume.

Throughout this specification, unless the context requires otherwise, the word “comprise”, or variations such as “comprises” or “comprising,” will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It must be noted that, as used in the subject specification, the singular forms “a,” “an,” and “the” include plural aspects unless the context clearly dictates otherwise. Thus, for example, reference to “an agent” includes a single agent, as well as two or more agents; reference to “the composition” includes a single composition, as well as two or more compositions; and so forth.

### BRIEF DESCRIPTION OF THE FIGURE

FIG. 1 is a flow chart illustrating an embodiment of the multiplex immunoassay comprising the following sequential and automated steps: (i) dewaxing of tissue section and epitope retrieval (exposing the epitopes on the tissue section); (ii) incubating tissue section with hydrogen peroxide (peroxidase block); (iii) incubating tissue section with a cocktail of primary mouse (Mab) and rabbit (Rab) antibodies; (iv) incubating tissue section with a horse radish peroxidase (HRP)-labelled anti-mouse antibody; (v) incubating tissue section with an alkaline phosphatase (AP)-labelled anti-rabbit antibody; (vi) incubating the tissue with 3,3 Diaminobenzidine (DAB) substrate/chromogen solution; (vii) incubating the tissue section with Fast Red substrate/chromogen solution (Red Refine 2); (viii) hematoxylin counterstaining the tissue section; and (ix) aqueously or permanently mounting the tissue section.

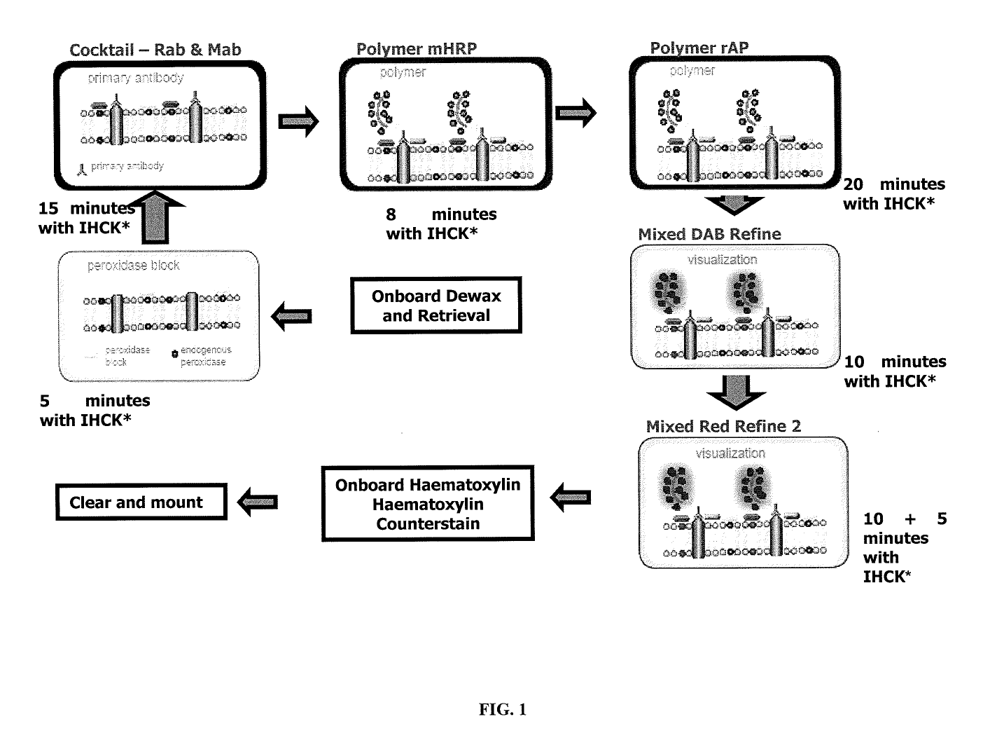

### DETAILED DESCRIPTION

In a first aspect, there is provided a method of detecting a plurality of epitopes in a biological tissue sample, the method comprising, in order, the following steps:
(a) providing a cross-section of a tissue sample attached to a solid substrate; wherein the tissue sample is embedded in an embedding medium;
(b) removing the embedding medium from the tissue cross-section of (a);
(c) incubating the tissue sample with two or more primary binding agents for a period of time sufficient to allow the two or more primary binding agents to bind to the plurality of epitopes, wherein two or more of the primary binding agents bind to different epitopes;
(d) incubating the tissue sample with two or more secondary binding agents for a period of time sufficient to allow the two or more secondary binding agents to bind to the primary binding agents, wherein each of the two or more of the secondary binding agents binds to a different primary binding agent and wherein two or more of the secondary binding agents comprise different detectable labels; and
(e) detecting the presence of the different detectable labels on the tissue sample, thereby distinguishing between the plurality epitopes;
   wherein progress from step (b) to (e) is automated and wherein the two or more primary binding agents and/or the two or more secondary binding agents are a cocktail of the binding agents.

Whilst not intending to limit the scope, methods are particularly suitable for immunohistochemical (IHC) analyses for detecting two or more target antigens on a tissue sample in situ. In an example of an IHC process, a biological sample is fixed and embedded in a suitable embedding medium and cut into thin cross-sections for staining and subsequent inspection by light microscopy. Sample processing steps for IHC may further include, for example, dewax, antigen retrieval, exposure to one or more primary antibodies (primary binding agent), washing, exposure to one or more secondary antibodies (secondary binding agent—each secondary antibody optionally coupled to a suitable detectable label), washing, applying a chromogen substrate specific for the detectable label(s), washing, counter staining (e.g., hematoxylin), applying a cover slip and examining the stained tissue section under light microscopy. Washing steps may be performed with any suitable buffer or solvent, e.g., phosphate-buffered saline, TRIS-buffered saline or distilled (or de-ionized) water. The wash buffer may optionally contain a surfactant. In certain embodiments the surfactant in one such as TWEEN™ 20 or NP-40.

Many types of biological tissue samples are compatible with the provided methods. Examples include tissue samples derived from living organism (e.g., an animal, such as mammals (e.g., humans), plants, fungi, archaea, or bacteria). Tissue samples may comprise one or more cells, such as a cell smear or colony, or a tissue specimen derived from an organ (e.g., biopsy material). In certain embodiments, the tissue sample is suspected of containing tumor cells or other cancer cells, including malignant cancer cells. In specific embodiments the tissue sample is from a biopsy performed on the subject or patient.

Tissue samples can be prepared by a variety of methods known to those skilled in the art, depending on the type of sample and the assay to be performed. For instance, tissue or cell samples may be fresh or preserved, and may be, for example, in liquid solution, flash-frozen or lyophilized, smeared or dried, embedded, or fixed on slides or other supports. In some embodiments, the tissue sample is prepared by fixing and embedding the tissue sample in a suitable embedding medium.

Fixing a tissue sample preserves the cells and other tissue constituents in as close to their natural state as possible so as to allow the tissue sample to withstand the subsequent stages of tissue processing, such as is required for immunohistochemical analysis and the like without significant changes to its composition or structure. Suitable methods of fixing a tissue sample will be known to those skilled in the art and can vary depending on the size, composition and structure of the tissue sample in question. For example, tissue samples can be fixed by perfusion or by submersion in a fixative solution. Suitable fixatives include, but are not limited to, cross-linking agents (such as aldehydes, e.g., formaldehyde, paraformaldehyde, and glutaraldehyde, as well as non-aldehyde cross-linking agents), oxidizing agents (e.g., metallic ions and complexes, such as osmium tetroxide and chromic acid), protein-denaturing agents (e.g., acetic acid, methanol, and ethanol), mercuric chloride, acetone, picric acid and reagents such as Carnoy's fixative, methacarn, Bouin's fluid, B5 fixative, Rossman's fluid and Gendre's fluid. In some embodiments, the tissue sample is fixed in formaldehyde, such as as 10% buffered formalin.

Once the tissue has been fixed, in certain embodiments it may be further preserved by embedding the tissue in an embedding medium. Suitable embedding media are known to those skilled in the art and includes, but are not limited to, paraffin, celloidin, OCT™ embedding compound, agar, plastics and acrylics. The process of embedding a tissue sample also allows the tissue to be sliced into thin cross-sections for mounting onto a solid substrate. Suitable solid supports are known to those skilled in the art, such as glass slides or other suitable planar support, which allow for microscopic inspection of the tissue section following immunohistochemical analysis and the like.

As many embedding media are hydrophobic in nature, they are not suitable for subsequent analyses that utilize hydrophilic reagents, such as IHC. Thus, the embedding medium needs to be removed prior to such analyses. The term “de-waxing” is broadly used herein to refer to the partial or complete removal of the embedding medium from the embedded tissue sample. Methods of de-waxing are know to those skilled in the art. For example, paraffin- embedded tissue sections can be de-waxed by passage through an organic solvent such as toluene, xylene or limonene.

In some embodiments, it may be useful to treat the tissue samples before contacting them with primary binding agents in order to increase the reactivity or accessibility of target antigens (or epitopes thereof) and to reduce non-specific interactions. Examples of suitable treatment protocols will be known to those skilled in the art and may include changes to buffer conditions, pH, pressure and temperature. Such treatment may also include more than one protocol, particularly when required to retrieve two different target antigens.

Methods for exposing or increasing the reactivity or accessibility of a target antigen to a primary binding agent are also referred to as “antigen retrieval,” “epitope retrieval,” or “unmasking”, examples of which are known to the skilled person. In some embodiments, epitope retrieval is achieved by enzymatic digestion of the tissue section with a proteolytic enzyme (e.g. proteinase, pronase, pepsin, papain, trypsin or neuraminidase) or by using heat (e.g. heat-induced epitope retrieval). Heating may involve microwave irradiation or a water bath, steamer, regular oven, autoclave or a pressure cooker in an appropriately pH stabilizing buffer, usually containing EDTA, EGTA, Tris-HCl, citrate, urea, glycin-HCl or boric acid. Surfactants may also be added to increase epitope retrieval, or added to the dilution media and/or rinsing buffers to lower non-specific binding. In some embodiments, combinations of different antigen retrieval methods may be used.

In some embodiments, epitope retrieval is achieved by (f) heating the tissue sample after step (b) and before step (c) in a composition comprising a buffer and a surfactant for a time sufficient to expose the plurality of epitopes and allow the two or more primary binding agents to bind to the plurality of epitopes, wherein progress from step (b) to (f) is automated. In some embodiments, heating the tissue sample during step (f) comprises heating the tissue sample to a temperature of from about 100° C. to about 102° C.

In some embodiments, epitope retrieval is achieved by (g) exposing the tissue sample after step (b) and before step (c) to a buffered enzyme solution for a time sufficient to expose the plurality of epitopes and allow the primary binding agents to bind to the plurality of epitopes, wherein progress from step (b) to (g) is automated.

As used herein, the term “binding agent” means any substance that is capable of recognizing (i.e., binding to) a target molecule. As used herein, the term “primary binding agent” means any substance that is capable of recognizing (i.e., binding to) a target antigen on the tissue sample, or an epitope thereof.

Suitable primary binding agents would be known to those skilled in the art and the choice will depend on the nature of the target antigen (or epitope thereof). In some embodiments, the primary binding agent is an antibody, or an antigen binding fragment thereof, also referred to herein as a primary antibody.

As used herein, the term “secondary binding agent” means any substance that is capable of binding to or otherwise recognizing a primary binding agent. Suitable secondary binding agents would be known to those skilled in the art. Examples include antibodies, or antigen binding fragments thereof, also referred to herein as secondary antibodies. The secondary binding agent may further comprise other functional elements, including, but not limited to, a polymer and/or linker segment, a detectable label, and/or an element that may be recognized by an adaptor unit or detectable label.

Antibodies suitable for use in accordance with described methods as binding agents would be known to those skilled in the art. Examples include, but are not limited to, polyclonal, monoclonal, monospecific, polyspecific, humanized, single-chain, chimeric, synthetic, recombinant, hybrid, mutated, and CDR-grafted antibodies. Various techniques for producing antibodies and preparing recombinant antibody molecules are known in the art. Antibodies may be derived from any species, including, but not limited to, rat, mouse, goat, guinea pig, donkey, rabbit, horse, lama, camel, or any avian species (e.g., chicken, duck). The antibody may be of any suitable isotype, such as IgG, IgM, IgA, IgD, IgE or any subclass thereof. The skilled addressee will appreciate that antibodies produced recombinantly, or by other means, for use in accordance with embodiments include antigen-binding fragments thereof that can still bind to or otherwise recognize the target molecule. Examples include Fab, an F(ab)₂, Fv, scFv fragments.

In some embodiments, the primary binding agent is a mouse immunoglobulin or an antigen binding fragment thereof, and the secondary binding agent is an anti-mouse antibody, or an antigen binding fragment thereof.

In some embodiments, the primary binding agent is a rabbit immunoglobulin, or an antigen binding fragment thereof, and the secondary binding agent is an anti-rabbit antibody or an antigen binding fragment thereof.

In some embodiments, the primary binding agent is selected from the group consisting of (i) a mouse anti-tyrosinase antibody or an antigen binding fragment thereof; (ii) a mouse anti-Melan A antibody, or an antigen binding portion thereof; (iii) a rabbit anti-S-100 antibody, or an antigen binding portion thereof; (iv) a mouse anti-CD3 antibody, or an antigen binding fragment thereof; (v) a rabbit anti-Ki67 antibody, or an antigen binding portion thereof; (vi) a mouse anti-CD20 antibody, or an antigen binding fragment thereof, and (vii) any combination thereof.

In some embodiments, the cocktail comprising two or more primary binding agents comprises a mouse anti-CD3 antibody, or an antigen binding fragment thereof, and a rabbit anti-Ki67 antibody, or an antigen binding portion thereof.

In some embodiments, the cocktail comprising two or more primary binding agents comprises a mouse anti-CD20 antibody, or an antigen binding fragment thereof, and a rabbit anti-Ki67 antibody, or an antigen binding portion thereof.

Additional methods may further comprise the use of third, forth, fifth, or even higher order, primary and/or secondary binding agents.

The terms “recognize,” “recognizing,” and the like, as used herein, mean an event in which one substance, such as a probe or binding agent, directly or indirectly interacts with a target molecule in such a way that the interaction with the target may be detected. In some examples, a probe may react with a target, or directly bind to a target, or indirectly react with or bind to a target by directly binding to another substance that in turn directly binds to or reacts with a target. The terms “specific for”, “specifically” and the like, as used herein in the context of describing binding between two or more entities, mean that the binding is through a specific interaction between complementary binding partners, rather than through non-specific aggregation.

In some embodiments, methods allow for the detection of at least two different target antigens on a tissue section (or epitopes thereof) by providing two or more primary binding agents and two or more secondary binding agents, each secondary binding agent comprising a different detectable label, wherein the detectable labels allow for distinguishing between a plurality of epitopes on the tissue sample to which the primary binding agents are bound.

In some embodiments, the two or more primary binding agents are in a cocktail of primary binding agents. In some embodiments, the two or more secondary binding agents are in a cocktail of secondary binding agents. In some embodiments, each of the two or more primary binding agents and the two or more secondary binding agents are in a cocktail of binding agents.

In some embodiments, where secondary binding agents are in a cocktail, the method will comprise incubating the tissue samples with the two or more primary binding agents in separate, automated steps. For example, the method of step (c) (i.e., incubating the tissue sample with two or more primary binding agents) can comprise, in order, the following steps:
(i) incubating the tissue sample with one of the two or more primary binding agents for a period of time sufficient to allow the primary binding agent to bind to an epitope;
(ii) incubating the tissue sample with another of the two or more primary binding agents for a period of time sufficient to allow the primary binding agent to bind to another epitope;
(iii) optionally repeating step (ii);
   wherein each of the primary binding agents of (i), (ii) and (iii) binds to a different epitope and wherein progress from steps (i) to (iii) is automated.

In some embodiments, where primary binding agents are in a cocktail, the method will comprise incubating the tissue samples with the two or more secondary binding agents in separate, automated steps. For example, the method of step (d) (i.e., incubating the tissue sample with two or more secondary binding agents) can comprise, in order, the following steps:
(i) incubating the tissue sample with one of the two or more secondary binding agents for a period of time sufficient to allow the secondary binding agent to bind to one of the two or more primary binding agents;
(ii) incubating the tissue sample with another of the two or more secondary binding agents for a period of time sufficient to allow the secondary binding agent to bind to another of the two or more primary binding agents;
(iii) optionally repeating step (ii) for each additional primary binding agent;
   wherein each of the secondary binding agents of (i), (ii) and (iii) binds to a different primary binding agent, wherein each of the secondary binding agents of (i), (ii) and (iii) comprises a different detectable label and wherein progress from steps (i) to (iii) is automated.

Where the method employs a cocktail of three primary binding agents, with each of the primary binding agents being specific for a different epitope constitutively expressed on the tissue sample, step (d) will comprise (i) incubating the tissue sample with a 1^{st} secondary binding agent for a period of time sufficient to allow the 1^{st} secondary binding agent to bind to the first of the three primary binding agents, (ii) incubating the tissue sample with a 2^{nd} secondary binding agent for a period of time sufficient to allow the 2^{nd} secondary binding agent to bind to the second of the three primary binding agents; (iii) incubating the tissue sample with a 3^{rd} secondary binding agent for a period of time sufficient to allow the 3^{rd} secondary binding agent to bind to the third of the three primary binding agents, wherein progress from steps (i) to (iii) is sequential and automated. Where there are four primary binding agents used, then step (d) will comprise an additional step (iv) of incubating the tissue sample with a 4^{th} secondary binding agent for a period of time sufficient to allow the 4^{th} secondary binding agent to bind to the additional (i.e., fourth) primary binding agent.

Separate and sequential incubation steps with the two or more secondary binding agents may be desirable where at least one of the secondary binding agents has a specific buffering requirement as compared to any of the other two or more secondary binding agents. Buffering conditions may depend, for example, on the type of detectable label that is being used. Preferred buffering conditions will be known to those skilled in the art.

In some embodiments, step (d) may comprises incubating the tissue sample with a cocktail of the two or more secondary binding agents (e.g., as a single incubation step). This may be desirable where the two or more secondary binding agents each have compatible buffering requirements, such that they can be used in a cocktail without adversely affecting their binding efficiency.

Suitable detectable labels are known to those skilled in the art and would include any molecule which may be detected directly or indirectly so as to reveal the presence of a target (e.g., epitope) in a tissue sample. Examples of detectable labels which may be used in accordance with methods and kits include enzymes, fluorophores, radioactive substances, chromophores, electrochemiluminescent labels, bioluminescent labels, polymers, polymer particles, bead or other solid surfaces, gold or other metal particles or heavy atoms, spin labels, radioisotopes, haptens, myc, nitrotyrosine, biotin, avidin, strepavidin, 2,4-dinitrophenyl, digoxigenin, bromodeoxy uridine, sulfonate, acetylaminoflurene, mercury trintrophonol and estradiol In some embodiments, a direct detectable label is used. Direct detectable labels may be detected per se without the need for additional molecules. In other embodiments, indirect detectable labels are used, which require the employment of one or more additional molecules. Examples include enzymes that affect a color change in a suitable substrate, as well as any molecule that may be specifically recognized by another substance carrying a label or react with a substance carrying a label.

Some detectable labels comprise “color labels” in which the target is detected by the presence of a color, or a change in color in the sample. Examples of “color labels” are chromophores, fluorophores, chemiluminescent compounds, electrochemiluminescent labels, bioluminescent labels and enzymes that catalyze a color change in a suitable substrate. In some embodiments, more than one type of color may be used, for instance, by attaching distinguishable color labels to more than one probe or binding agent, each carrying a different and distinguishable color label.

In some embodiments, the detectable label is an enzyme. Suitable enzymes are known to those skilled in the art. Examples of enzymes which may be used in accordance with methods and kids include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO). In some embodiments, the detectable labels are HRP and AP.

In some embodiments, molecular conjugates can be employed that couple the detectable label to a secondary binding agent via a polymeric carrier directly, or indirectly through reactive functional groups. Suitable polymeric carriers include, but are not limited to, biotin and non-biotin polymers.

Methods of forming a molecular conjugate may comprise providing a polymeric carrier comprising a polymeric portion selected from polyacrylic acids, polyacrylamide-N-hydroxysuccinimide, polyethyleneimines, polysaccharides, polyethylene-alt-maleic acids, polyamino acids, polyvinylpyrrolidones or polyguanidine. The polymeric portion may also include plural reactive functional groups selected from hydrazines, hydrazides, hydrazine derivatives, hydrazide derivatives, guanidines, aminoguanidines, hydroxyl amines, or combinations thereof. Exemplary polysaccharide species include carbohydrates, cellulose, carboxymethylcellulose, dextran, glycogen, polyhyaluronic acid and starch.

In one embodiment, the molecular conjugate is BOND™ Polymer Refine Detection system (Leica Microsystems). BOND™ Polymer Refine Detection system is a biotin-free, polymeric horseradish peroxidase (HRP)-linker antibody conjugate system.

In some embodiments, the detectable label is an enzyme that is detected by exposure to a suitable substrate (chromogen) that produces a color change that can be visualized by light microscopy or other suitable means of visualization/detection known to those skilled in the art. Examples of suitable substrates for horse radish peroxidase (HRP) include, but are not limited to, 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (α-NP), o-dianisidine (OD), 5-bromo-4-chloro-3-indolylphosphate (BCIP), Nitro blue tetrazolium (NBT), 2-(p-iodophenyl)-3-p-nitrophenyl-5-phenyl tetrazolium chloride (INT), tetranitro blue tetrazolium (TNBT), 5-bromo-4-chloro-3-indoxyl-beta-D-galactoside/ferro-ferricyanide (BCIG/FF). Examples of suitable substrates for Alkaline Phosphatase include, but are not limited to, Naphthol-AS-B 1-phosphate/fast red TR (NABP/FR), Naphthol-AS-MX-phosphate/fast red TR (VAMP/FR), Naphthol-AS-B1-phosphate/fast red TR (NABP/FR), Naphthol-AS-MX-phosphate/fast red TR (NAMP/FR), Naphthol-AS-Bl-phosphate/new fuschin (NABP/NF), bromochloroindolyl phosphate/nitroblue tetrazolium (BCIP/NBT), 5-Bromo-4-chloro-3-indolyl-b(beta)-d (delta)-galactopyranoside (BCIG).

In some embodiments, the method employs suitable chromogens simultaneously, for example, as a cocktail of chromogens. For instance, where a method employs a secondary binding agent comprising HRP and the another secondary binding agent comprising Alkaline Phosphatase, the cocktail can comprise 3,3′-diaminobenzidine (DAB) and Naphthol-AS-B1-phosphate/fast red TR (NABP/FR). In some embodiments, the chromogens can be used sequentially, for example, each chromogen applied to the tissue section in separate, automated steps.

In some embodiments, signal amplification may allow for 1 up to 500 detectable label molecules per primary binding agent. For example, a primary antibody may be contacted with a secondary antibody conjugated to multiple detectable labels. In some embodiments, the detectable label is an enzyme conjugated to a polymer, such that the number of enzyme molecules conjugated to each polymer molecule is, for instance, 1 to 200, 2 to 50, or 2 to 25.

Any two or more compatible enzymes can be employed in methods as long as the enzymes yield pigments (stains) of distinct but inherently mergeable colors following exposure to suitable substrates. This means that each enzyme affords a pigment having a distinct color, but when co-localized the pigments together yield a distinctive intermediate color that is viewable or detectable in a single image. By way of example, an enzyme may afford a pigment having a red color, the other enzyme may afford a pigment having a blue color, and when co-localized the two pigments yield an intermediate purple color.

The use of the plurality of enzymes as detectable labels is intended to be carried out in the absence of background staining or contrasting stains that might interfere with observation or detection of the resulting pigments or stains. However, where enzymes are used as detectable labels, there are often problems with non-specific color changes (i.e., staining) arising from the presence of endogenous enzymes within the tissue sample. Methods for eliminating or reducing the level of non-specific staining arising from endogenous enzymes will be known to those skilled in the art. For example, endogenous biotin and peroxidase activity may be removed by treatment with hydrogen peroxide, while endogenous phosphatase activity may be removed by treatment with levamisole. Heating the tissue sample may also be used destroy endogenous phosphatase and esterase activity.

In some embodiments, non-specific staining is reduced by (h) exposing the tissue sample to hydrogen peroxide after step (b) and before step (c) for a time sufficient to exhaust any endogenous peroxidase in the tissue sample and minimize non-specific staining during step (e), wherein progress from step (b) to (h) is automated.

In some embodiments, additional treatments may be performed to reduce non-specific binding of the primary and/or secondary binding agents to the tissue sample. For example, carrier proteins, carrier nucleic acid molecules, salts, or detergents may reduce or prevent non-specific binding. Non-specific binding sites may be blocked in some embodiments with inert proteins like, HSA, BSA, ovalbumin, fetal calf serum or other sera, or with detergents like TWEEN™20, TRITON™ X-100, Saponin, BRIJ™, or PLURONICS™. Alternatively, non-specific binding sites may be blocked with unlabeled competitors for the recognition event between the target antigen and binding agents. Salt, buffer and temperature conditions may also be modified so as to reduce non-specific binding.

Where the detectable label facilitates a color change, this can be qualitatively assessed by the user under light microscopy or other suitable means know to those skilled in the art. For example, analysis of the specific cell populations may include a step of determining the portion of cells of that exhibit one or both detectable labels, as evidenced, for example, by the presence of a color change specific for a particular detectable label (i.e., a stain or pigment). For example, one calculation may involve assessing the portion of cells that display two different stains relative to the sum of those that display both stains and those that display only one of the two stains. Alternatively, another calculation may involve assessing the portion of cells that display only one stain relative to the sum of those that display both stains and those that display only the one stain.

In some embodiments, an approximation of the amount of a target in a sample can be determined from the intensity and localization of the color change on a tissue sample. The intensity of the color from the sample may also be compared to that of a known standard or control tissue sample. Estimating the amount of a detectable target in a sample is helpful, for instance, in a variety of diagnostic tests, and the estimate may be used to plan a course of treatment for a suspected disease or condition. Several commercial densitometry software programs and related instruments are also available to quantitate the intensity and/or distribution of a color change on a tissue sample.

Methods can be used for the detection or monitoring of diseases or conditions (e.g., those associated with changes to the number of cells or their phenotype), including viral infections, autoimmune diseases, melanoma, lymphoma and other cancers. The automated methods can be used for monitoring of treatment efficacy.

In some embodiments, methods are compatible with automated staining protocols and equipment. Suitable equipment for performing methods will be known to those skilled in the art. Examples include the BenchMark staining platforms (Ventana) and the BOND™ tissue staining instrument (Leica Microsystems).

The term “automated”, as used herein, means that progression from one step of the method to a following step is automatically controlled (e.g., without direct user intervention). For example, the exposure of the tissue samples to reagents and solutions can be controlled by a machine with appropriate software telling the machine when and how much of the reagents or solutions to dispense onto the tissue sections and providing instructions to progress the tissue sections from one part of the method to the next. For instance, the machine can be programmed to dewax the tissue sample(s), wash, dispense primary binding agents, wash, dispense secondary binding agents (as a single step or as multiple sequential steps), wash, etc.

An automated system differs from a manual system in that the latter will require operator intervention (e.g., between any two steps). For example, in a manual system, the operator must remove the tissue sections from the dewaxing solution, apply a wash to each tissue section, optionally perform an antigen retrieval step to expose the epitopes of interest, apply a wash, optionally apply a blocking agent to avoid or minimize any non-specific binding and/or staining, apply a wash, apply the primary binding agents, etc, thus requiring the operator to handle the tissue sections between the various steps of the process. By contrast, an automated system only requires the operator to handle the tissue section before the commencement of the method (i.e., set-up) and once the method has been completed (i.e., for detection). An automated system is therefore advantageous because it can reduce the time and cost of performing the method, which can have a significant economic benefit to the provision of health care in the community.

The total time necessary to complete steps (b) to (e), (b) to (0, (b) to (g) or (b) to (h) may depend on various factors, including, but not limited to, the type of primary and secondary binding agents used and the time required for optimal epitope retrieval, peroxidase block and chromogen exposure. Wash times are also variable. Those skilled in the art are able to determine the time required for each step in automated methods, taking into account some of the aforementioned factors and others. Tables 1, 2 and 3 illustrate examples of the times required for the automated steps of various embodiments as compared to other methods that do not employ reagent cocktails, for example, the method illustrated in Table 4 (note: heat pre-treatment, washes and haematoxylin counterstain times are not shown. A haematoxylin counterstain step may add a further 2-5 minutes. Wash times are variable):

TABLE 1
Parallel Staining (6 washes)

| | | Min Time | Max Time |
|---|---|---|---|
| Protocol | Reagents | (mins) | (mins) |
| One | Dewax | 0.5 | 0.5 |
| Protocol | Primary Ab Cocktail | 15 | 60 |
| | 1^{st} Secondary Ab | 8 | 8 |
| | 2^{nd} Secondary Ab | 20 | 20 |
| | 1^{st} Chromogen | 15 | 15 |
| | 2^{nd} Chromogen | 10 | 10 |
| | Time | 68.5 | 113.5 |

TABLE 2
Parallel Staining (6 washes)

| | | Min Time | Max Time |
|---|---|---|---|
| Protocol | Reagents | (mins) | (mins) |
| One | Dewax | 0.5 | 0.5 |
| Protocol | 1^{st} Primary | 15 | 60 |
| | Ab | | |
| | 2^{nd} Primary | 15 | 60 |
| | Ab | | |
| | Secondary | 8 | 20 |
| | Ab Cocktail | | |
| | 1^{st} | 15 | 15 |
| | Chromogen | | |
| | 2^{nd} | 10 | 10 |
| | Chromogen | | |
| | Time | 63.5 | 165.5 |

TABLE 3
Parallel Staining (5 washes)

| | | Min Time | Max Time |
|---|---|---|---|
| Protocol | Reagents | (mins) | (mins) |
| One | Dewax | 0.5 | 0.5 |
| Protocol | Primary Ab Cocktail | 15 | 60 |
| | Secondary Ab Cocktail | 8 | 20 |
| | 1^{st} Chromogen | 15 | 15 |
| | 2^{nd} Chromogen | 10 | 10 |
| | Time | 48.5 | 105.5 |

TABLE 4
Sequential Staining (7 washes)

| | | Min Time | Max Time |
|---|---|---|---|
| Protocol | Reagents | (mins) | (mins) |
| 1 | Dewax | 0.5 | 0.5 |
| | 1^{st} Primary Ab | 15 | 60 |
| | 1^{st} Secondary Ab | 8 | 8 |
| | 1^{st} Chromogen | 10 | 10 |
| 2 | 2^{nd} Primary Ab | 15 | 60 |
| | 2^{nd} Secondary Ab | 20 | 20 |
| | 2^{nd} Chromogen | 15 | 15 |
| | Time | 83.5 | 173.5 |

Table 5 provides a further example of the times required for the automated steps in certain embodiments, including washing steps, epitope retrieval (ER) and peroxidase block:

TABLE 5

| | | | |
|---|---|---|---|
| Step | | Min Time | Max Time |
| Number | Reagent Name | (mins) | (mins) |

Dewaxing

| | | | |
|---|---|---|---|
| 1 | Bond Dewax | 0.5 | 0.5 |
| | Solution | | |
| 2 | Bond Dewax | 0 | 0 |
| | Solution | | |
| 3 | Bond Dewax | 0 | 0 |
| | Solution | | |
| 4 | Alcohol | 0 | 0 |
| 5 | Alcohol | 0 | 0 |
| 6 | Alcohol | 0 | 0 |
| 7 | Bond Wash | 0 | 5 |
| | Solution | | |
| 8 | Bond Wash | | |
| | Solution | | |
| 9 | Bond Wash | 5 | 15 |
| | Solution | | |
| | Total Time | 5.5 | 20.5 |

Epitope Retrieval (ER; optional)

| | | | |
|---|---|---|---|
| 1 | Bond ER | 0 | 15 |
| | Solution 1 | | |
| 2 | Bond ER | 0 | 15 |
| | Solution 1 | | |
| 3 | Bond ER | 20 | 60 |
| | Solution 1 | | |
| 4 | Bond ER | 12 | 12 |
| | Solution 1 | | |
| 5 | Bond Wash | 10 | 10 |
| | Solution | | |
| 6 | Bond Wash | 0 | 10 |
| | Solution | | |
| 7 | Bond Wash | 0 | 15 |
| | Solution | | |
| 8 | Bond Wash | 3 | 15 |
| | Solution | | |
| | Total Time | 45 | 152 |

Staining Protocol

| | | | |
|---|---|---|---|
| 1 | Peroxide Block | 0 | 0 |
| 2 | Bond Wash | 0 | 0 |
| | Solution | | |
| 3 | Bond Wash | 0 | 0 |
| | Solution | | |
| 4 | Bond Wash | 0 | 15 |
| | Solution | | |
| 5 | PRIMARY Ab | 15 | 60 |
| | cocktail | | |
| 6 | Bond Wash | 0 | 0 |
| | Solution | | |
| 7 | Bond Wash | 0 | 0 |
| | Solution | | |
| 8 | Bond Wash | 0 | 15 |
| | Solution | | |
| 9 | Polymer mHRP | 8 | 8 |
| 10 | Bond Wash | 0 | 0 |
| | Solution | | |
| 11 | Bond Wash | 0 | 0 |
| | Solution | | |
| 12 | Bond Wash | 0 | 15 |
| | Solution | | |
| 13 | Polymer rAP | 20 | 20 |
| 14 | Bond Wash | 0 | 0 |
| | Solution | | |
| 15 | Bond Wash | 0 | 0 |
| | Solution | | |
| 16 | Bond Wash | 5 | 15 |
| | Solution | | |
| 17 | Bond Wash | 0 | 0 |
| | Solution | | |
| 18 | Bond Wash | 0 | 15 |
| | Solution | | |
| 19 | Deionized Water | 0 | 15 |
| 20 | Mixed DAB | 0 | 0 |
| | Refine | | |
| 21 | Mixed DAB | 10 | 10 |
| | Refine | | |
| 22 | Deionized Water | 0 | 0 |
| 23 | Deionized Water | 0 | 0 |
| 24 | Deionized Water | 0 | 30 |
| 25 | Mixed Red | 10 | 10 |
| | Refine 2 | | |
| 26 | Mixed Red | 5 | 0 |
| | Refine 2 | | |
| 27 | Deionized Water | 0 | 0 |
| 28 | Deionized Water | 0 | 0 |
| 29 | Deionized Water | 0 | 15 |
| 30 | Hematoxylin | 5 | 5 |
| | DS9477 | | |
| 31 | Deionized Water | 0 | 5 |
| 32 | Bond Wash | 0 | 5 |
| | Solution | | |
| 33 | Deionized Water | 0 | 0 |
| | Total Time | 78 | 258 |

In some embodiments, the total time necessary to complete steps (b) to (e), (b) to (f), (b) to (g) or (b) to (h) of the automated methods is less than about 4 hours. In certain embodiments, the total time for completing steps (b) to (e), (b) to (f), (b) to (g) or (b) to (h) of the automated methods is between about, no more than about, or no less than about 1.5, 1.75, 2.0, 2.25, 2.5, 2.75, 3.0, 3.25, 3.5, 3.75, 4.0, 4.25, 4.5, 4.75, or 5.0 hours, or any range derivable therein.

Those skilled in the art will appreciate that the embodiments described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments will now be described with reference to the following examples which are intended for the purpose of illustration only and are not intended to limit the scope of the generality hereinbefore described.

### EXAMPLES

### Materials And Methods

Labelled slides containing formalin-fixed, paraffin-embedded tissue sections are placed on a BOND™ fully automated immunohistochemistry instrument (Leica Microsystems). The label contains information that is recognised by and instructs the instrument to carry out steps 1, 2 and 3, as outlined below.

### Step 1—Dewax Protocol

To remove paraffin wax and rehydrate the tissue section(s), BOND™ Dewax Solution (a solvent-based solution) is applied to heated slides. Alcohol is then applied to the slides to remove the BOND™ Dewax Solution. BOND™ Wash Solution (a Tris-buffered saline solution comprising a surfactant and 0.35% ProClin™ 950) is then applied to slides to remove the alcohol and rehydrate the tissue section.

### Step 2 (Optional)—Antigen Retrieval

The dewax protocol of Step 1 is optionally followed by Steps 2A or 2B. The choice of method for antigen retrieval will invariably depend on the primary antibodies (primary binding agents) that will be used and, hence, the nature of the target antigen(s) (biomarker(s)). In some instances, the biomarker of interest will need to be retrieved (i.e., exposed) to allow for binding with the first or second probe specific to that biomarker.

### A. Heat Pre-Treatment Protocol

Without being bound by theory, exposing the sectioned tissue to a heated buffer solution results changes the conformation of the tissue structure and exposes the antigens of interest, thus improving staining. BOND™ Epitope Retrieval Solution 1 (a citrate based buffer and surfactant) or BOND™ Epitope Retrieval Solution 2 (an EDTA based buffer and surfactant) is applied to heated slides and incubated for a period of time sufficient to retrieve the antigens of interest. The incubation time and temperature may vary depending on, for example, the nature of the antigen in question and the extent of tissue fixation. BOND™ Wash Solution (a Tris-buffered saline solution comprising a surfactant and 0.35% ProClin™ 950) is then applied to the slides to remove the BOND™ Epitope Retrieval Solution.

### B. Enzyme Pre-Treatment Protocol

An enzyme solution comprising a proteolytic enzyme in a Tris-buffered saline solution (also comprising a stabilizer, a surfactant and 0.35% ProClin™ 950) is applied to heated slides and incubated for a period of time sufficient to retrieve the antigens of interest. The incubation time, temperature and enzyme concentration may vary depending on, for example, the nature of the antigen in question and the extent of tissue fixation. BOND™ Wash Solution is then applied to the slides to remove the enzyme solution.

### Step 3—Staining Protocol

Peroxide Block (3-4% Hydrogen peroxide) is applied to slides and incubated for a period of time sufficient to reduce unwanted background staining that may arise as a result of endogenous peroxidase that may be present in the tissue sample. BOND™ Wash Solution™ is then applied to slides to remove excess Peroxide Block.

A primary antibody cocktail comprising mouse immunoglobulin to a first epitope (1^{st} biomarker) and a rabbit immunoglobulin to a second epitope (2^{nd} biomarker) is applied to the tissue sections and incubated for a period of time sufficient to allow the immunoglobulins to bind to their respective epitopes. The incubation time will depend upon the primary antibodies, the nature of the biomarkers and the degree of exposure of the epitopes in question. BOND™ Wash Solution is then applied to slides to remove excess primary antibodies.

A horse radish peroxidase (HRP)-labelled, anti-mouse antibody in a solution comprising 10% (v/v) animal serum in Tris-buffered saline and 0.09% ProClin™ 950 is applied to tissue sections and incubated for a period of time sufficient to enable the poly-mouse HRP antibody to bind to the mouse immunoglobulin that is, in turn, bound to the 1^{st} biomarker. BOND™ Wash Solution is then applied to the tissue sections to remove any unbound poly-HRP anti-mouse antibodies.

An alkaline phosphatase (AP)-labelled, anti-rabbit antibody in a solution comprising 10% (v/v) animal serum in Tris-buffered saline and 0.09% ProClin 950 is applied to the tissue sections and incubated for a period of time sufficient to enable the poly-AP antibody to bind to the rabbit immunoglobulin that is, in turn, bound to the 2^{nd} biomarker. BOND™ Wash Solution is then applied to the tissue sections to remove any unbound poly-AP anti-rabbit antibodies.

De-ionized water is then applied to the tissue sections to remove the BOND™ Wash Solution. 3,3 Diaminobenzidine (DAB) substrate/chromogen solution is then applied to the tissue sections and incubated for a period of time sufficient to produce a brown precipitate following a reaction with the HRP. The brown precipitate can be visualised by light microscopy and is indicative of the presence of the 1^{st} biomarker.

De-ionized water is then applied to the tissue section to remove the DAB solution and a Fast Red substrate/chromogen solution is applied to the tissue sections and incubated for a period of time sufficient to produce a red precipitate following a reaction with the AP. The red precipitate can be visualised by light microscopy and is indicative of the presence of the 2^{nd} biomarker.

De-ionised water is then applied to the tissue sections to remove the Fast Red substrate/chromogen solution. Hematoxylin is applied to the tissue sections and incubated for a period of time sufficient to stain the nuclei to the extent they can be visualised by light microscopy. De-ionized water is applied to slides to remove excess hematoxylin. BOND™ Wash Solution can then be applied to the tissue sections to remove the de-ionised water and, if necessary, intensify the ‘blue’ nuclei staining. De-ionized water is then applied to tissue sections to remove the BOND™ Wash Solution.

The slides are then removed from the automated immunohistochemistry instrument and either aqueously or permanently mounted. AMENDMENT AMENDMENTS TO THE CLAIMS

1. A method of detecting a plurality of epitopes in a biological tissue sample, the method comprising, in order, the following steps:
(a) providing a cross-section of a tissue sample attached to a solid substrate; wherein the tissue sample is embedded in an embedding medium;
(b) removing the embedding medium from the tissue cross-section of (a);
(c) incubating the tissue sample with two or more primary binding agents for a period of time sufficient to allow the two or more primary binding agents to bind to the plurality of epitopes, wherein two or more of the primary binding agents bind to different epitopes;
(d) incubating the tissue sample with two or more secondary binding agents for a period of time sufficient to allow the two or more secondary binding agents to bind to the primary binding agents, wherein each of the two or more of the secondary binding agents binds to a different primary binding agent and wherein two or more of the secondary binding agents comprise different detectable labels; and
(e) detecting the presence of the different detectable labels on the tissue sample, thereby distinguishing between the plurality epitopes;
wherein progress from step (b) to (e) is automated and wherein the two or more primary binding agents and/or the two or more secondary binding agents are a cocktail of the binding agents.

2. The method of claim 1, wherein the two or more primary binding agents are a cocktail of primary binding agents.

3. The method of claim 1, wherein the two or more secondary binding agents are a cocktail of secondary binding agents.

4. The method of claim 1, comprising (f) heating the tissue sample after step (b) and before step (c) in a solution comprising a buffer and a surfactant for a time sufficient to expose the plurality of epitopes and allow the two or more primary binding agents to bind to the plurality of epitopes.

5. The method of claim 1, comprising (g) exposing the tissue sample after step (b) and before step (c) to a buffered enzyme solution for a time sufficient to expose the plurality of epitopes and allow the two or more primary binding agents to bind to the plurality of epitopes.

6. The method of claim 1, comprising (h) exposing the tissue sample to hydrogen peroxide after step (b) and before step (c) for a time sufficient to exhaust any endogenous peroxidase in the tissue sample and minimize non-specific detection of a detectable label during step (e).

7. The method of claim 1, wherein the two or more primary binding agents comprise a mouse immunoglobulin, or an antigen binding fragment thereof, and a rabbit immunoglobulin, or an antigen binding fragment thereof.

8. The method of claim 7, wherein the two or more secondary binding agents comprise an anti-mouse antibody, or an antigen binding fragment thereof, and an anti-rabbit antibody, or an antigen binding fragment thereof.

9. The method of claim 7, wherein the two or more primary binding agents comprise a mouse anti-tyrosinase antibody, or an antigen binding fragment thereof.

10. The method of claim 7, wherein the two or more primary binding agents comprise a mouse anti-Melan A antibody, or an antigen binding fragment thereof.

11. The method of claim 7, wherein the two or more primary binding agents comprise a rabbit anti-S-100 antibody, or an antigen binding portion thereof.

12. The method of claim 10, wherein the two or more primary binding agents comprise a rabbit anti-S-100 antibody, or an antigen binding portion thereof.

13. The method of claim 7, wherein the two or more primary binding agents comprise a mouse anti-CD3 antibody, or an antigen binding fragment thereof.

14. The method of claim 7, wherein the two or more primary binding agents comprise a mouse anti-CD20 antibody, or an antigen binding fragment thereof.

15. The method of claim 13, wherein the two or more primary binding agents comprise a rabbit anti-Ki67 antibody, or an antigen binding portion thereof.

16. The method of claim 1, wherein the solid substrate is a histology slide that is in continuous contact with a slide holder of an automated immunostainer during steps (b) to (e).

17. The method of claim 1, wherein steps (b) to (e) are completed in less than about 4 hours.

18. The method of claim 2, wherein step (d) comprises, in order, the following steps:
(i) incubating the tissue sample with one of the two or more secondary binding agents for a period of time sufficient to allow the secondary binding agent to bind to one of the two or more primary binding agents;
(ii) incubating the tissue sample with another of the two or more secondary binding agents for a period of time sufficient to allow the secondary binding agent to bind to another of the two or more primary binding agents;
(iii) optionally repeating step (ii) for each additional primary binding agent;
wherein each of the secondary binding agents of (i), (ii) and (iii) binds to a different primary binding agent, wherein each of the secondary binding agents of (i), (ii) and (iii) comprises a different detectable label and wherein progress from steps (i) to (iii) is automated.

19. The method of claim 3, wherein step (c) comprises, in order, the following steps:
(ii) incubating the tissue sample with one of the two or more primary binding agents for a period of time sufficient to allow the primary binding agent to bind to an epitope;
(ii) incubating the tissue sample with another of the two or more primary binding agents for a period of time sufficient to allow the primary binding agent to bind to another epitope;
(iii) optionally repeating step (ii);
wherein each of the primary binding agents of (i), (ii) and (iii) binds to a different epitope and wherein progress from steps (i) to (iii) is automated.

20. The method of claim 9, wherein the two or more primary binding agents comprise a mouse anti-Melan A antibody, or an antigen binding fragment thereof.

21. The method of claim 9, wherein the two or more primary binding agents comprise a rabbit anti-S-100 antibody, or an antigen binding portion thereof.

22. The method of claim 14, wherein the two or more primary binding agents comprise a rabbit anti-Ki67 antibody, or an antigen binding portion thereof.
